(19) United States
(12) Patent Application Publication
(10) Pub. No.: US 2023/0132309 A1
(43) Pub. Date: Apr. 27, 2023

(54) Utilizing Resonant Frequency in Vibration Therapy

(71) Applicant: Omar Afifeh, Roseville, CA (US)

(72) Inventor: Omar Afifeh, Roseville, CA (US)

(21) Appl. No.: 17/510,540

(22) Filed: Oct. 26, 2021

(51) Int. Cl. A61H 1/00

(52) U.S. Cl. CPC A61H 1/008; A61H 1/005; A61H 2201/1635; A61H 2201/164; A61H 2201/1623

(57) ABSTRACT

Vibration therapy delivered as a low-magnitude; high-frequency stimulus offers a means to deliver relevant mechanical signals safely to patients who cannot exercise to build musculoskeletal strength. Vibrations will begin at the cellular level and metastasize to the muscles and the entire musculoskeletal system. What I am proposing here is instead of using mechanical loading, we can do it more effectively using “Resonant Frequency”; which is the frequency of an external oscillation or vibration that matches an object (or cavity's) natural frequency, as a result either causes it to vibrate or increases its amplitude of oscillation. In mechanical systems, resonance refers to the amplification, reinforcement, or prolongation of sound or other vibrations. Using an external oscillation equipment, we match the natural frequency for the musculoskeletal system which will create vibration throughout that system which will improve bone density, increase strength and increased mobility.

### BACKGROUND

Resonance is the phenomenon at which the natural frequency of a system coincides with the driving periodically applied force on that system and gives the maximum response. The resonant frequency is the frequency at which resonance happens. There are many conditions associated with reduced mobility and overall system decline. This typically leads to failure of the musculoskeletal system with loss of skeletal strength and muscle dysfunction significantly compromising measures of life quality as such, pathological or occupational reductions in functional loading manifest as bone becomes more susceptible to fracture. According to studies, gentle vibration can improve bone density and prevent fractures which is why doctors use Vibration Therapy for the bones and muscles to improve mobility. So far, this procedure is done using mechanical loading. Vibration therapy delivered as a low-magnitude; high-frequency stimulus offers a means to deliver relevant mechanical signals safely to patients who cannot exercise to build musculoskeletal strength. It is important to point out that these vibrations will begin at the cellular level which are felt by neurons and metastasize to the muscles and the entire musculoskeletal system.

### SUMMARY

Instead of using mechanical loading, we can do it more effectively using “Resonant Frequency”; which is the frequency of an external oscillation or vibration that matches an object (or cavity's) natural frequency, and as a result either causes it to vibrate or increases its amplitude of oscillation. In mechanical systems, resonance refers to the amplification, reinforcement, or prolongation of sound or other vibrations. Using an external oscillation equipment, we match the natural frequency for the musculoskeletal system which will create vibration throughout that system which will improve bone density, increase strength, and eventually leads to increased mobility.

### BRIEF DESCRIPTION OF DRAWINGS

FIG. 1 Shows Power Source generating current to belt used to generate mechanical v vibrations through a #12 wire (combinedly referred to as ‘the device’).

FIG. 2 shows the device connected to elbow joint and part of the humerus, the radius and ulna bones.

FIG. 3 shows the device connected to the radius and ulna bones for therapy

FIG. 4 shows the device connected to the tibia and fibula bones

FIG. 5 shows the device connected to the spinal column, vertebras and lower back muscles

FIG. 6 shows the device connected to the femur

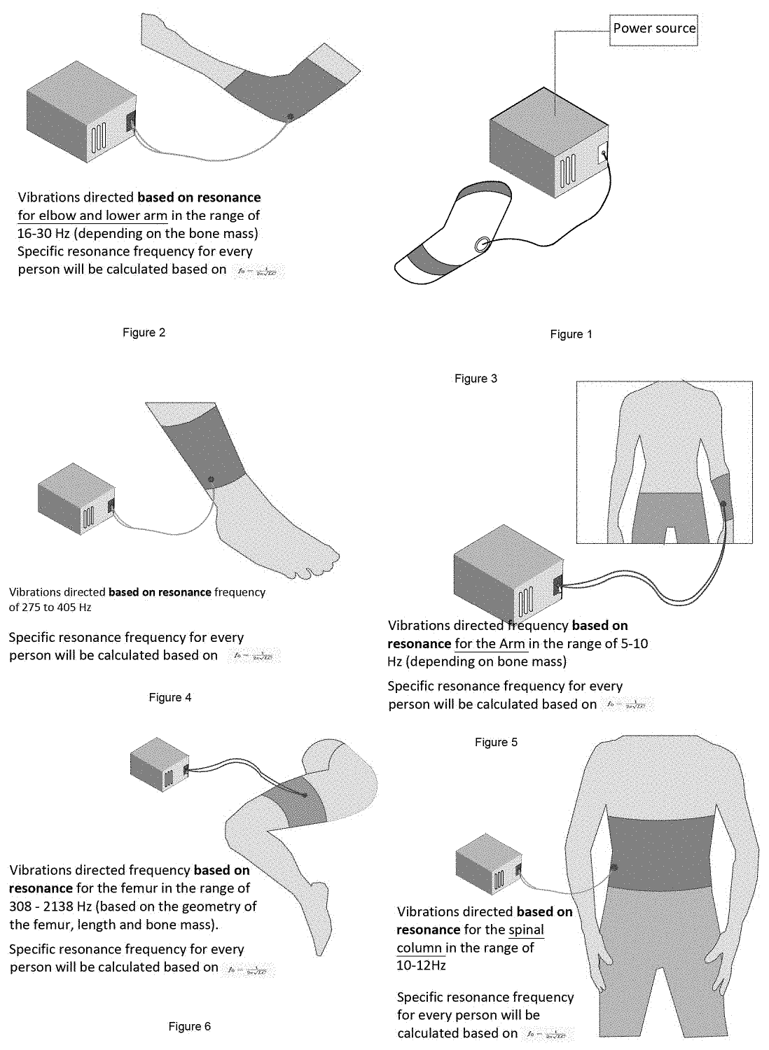

### DETAILED DESCRIPTION OF INVENTION

This patent focuses on frequencies equal to resonant frequency of human bones resulting from vibrations generated by external device and its effect on healing bone fracture when applied on human bones such as the femur. Whole body vibration through creating oscillation generating frequency equal to the critical bone resonant frequency.

### Bone Tissue's Resonance Frequency

Therapy by vibration utilized vibration to mechanically stimulate different organs in the body which leads to health improvements such as better movement and increased density for these organs. As illustrated above, each organ naturally has its own unique resonance frequency so we must adjust the vibration frequency and amplitude depending on the body part we're treating and applying the vibration to achieve best outcome through resonance effect. These frequencies are not to be confused with the speed of the vibration induced by the equipment. While conducting this method, when we are treating a certain body part at its specific corresponding frequency by using a machine to induce these vibrations, that organ or body part can achieve the highest responding vibration amplitude at that natural (or resonance) frequency which is called “resonance”.

There are multiple factors that determine the natural frequency of the organ such as its physical composition, mass, size and rigidity. The lighter and more rigid the organ, the higher its natural frequency while a relatively larger and more flexible organ will have a lower natural or resonance frequency. Human body-as-a-whole has a lower natural frequency between 5 Hz and 10 Hz, it varies based on the human's weight and how rigid (muscular) or flexible it is. Obviously, the density changes flexibility of the same body changes depending on the state of the body (the position, movement how relaxed or intense the muscles are . . . etc.).

### Utilizing Resonance Frequency in Vibration Therapy for Bones & Skeletal Muscles

Through the natural frequency of different body parts, our objective is to vibrate the skeletal muscles of the targeted organ to generate muscle contraction without resorting to higher vibrations or higher frequency but instead using the frequency that each origan will respond to best. If for example we are targeting the leg, it is a relatively denser bone, so we will utilize a lower frequency of 2-20 Hz but if we're treating the hand, we will be applying vibrations with frequencies between 30 and 50 Hz. We will apply different frequency vibrations to different body parts. Skeletal muscles have unique response to natural frequency, i.e. when they are subjected to vibration, the muscle tissues contract, and therefore increase rigidity which results in increasing their natural frequency. Skeletal muscles are naturally poised to maintain higher natural frequency than the external vibration frequency. Naturally, our bodies maintain their sizes by controlling the skeletal muscles' length. When skeletal muscles are stretched, they immediately contract as a reflex. As mentioned above, when these muscles contract, they become denser i.e., rigid, consequently, their natural frequencies increase as if they are resisting resonance to prevent overstretching. This is exactly what happens when these muscles are subjected to vibration, skeletal muscles always react to refuse resonance, skeletal muscles increase their natural frequencies by contracting to remain above external vibration frequency, or in other words, they try to stay out of resonance state. We will utilize that characteristic of the skeletal muscles to conduct our therapy but instead of using higher frequency vibration, we will focus vibrations with frequencies equal to the resonance or natural frequency, i.e. we will match the mechanical frequency directed on the body part being treated to the natural (resonance) frequency of that body part.

The following procedural specifications and modifications must be implemented:

Vibration device with stimulation electrode will be placed on the specific body part being treated. (13)

Stimulation electrode emits waves with frequency equal to the resonant frequency of the bone or part being treated (difference from other patented vibration therapies). (14)

Process is repeated on ongoing basis as recommended by the patient's physician. (15)

Depending on the shape of the bone (Oregon) being treated, the device applying the stimulation vibrations or waves shall take the shape of that surface. (16)

Since the natural frequency of the skeletal muscle ranges from 10 Hz (when they are relaxed) to 50 Hz when they are most contracted, the vibration device must track and increase the vibration frequency accordingly to maintain its contraction attempts to remain above resonance gradually and continuously. Unlike existing treatment exposing skeletal muscle tissues to constant high frequency. At no point of the treatment, shall the vibration frequency be lower than what is required to trigger natural contraction reflex response. Continuous and rapidly increasing skeletal muscle contraction causes mechanical stimulation to the human body which consequently activates biological activities and tissues which positively influences the cellular processes.

1. The effect of vibrations with frequencies equal to the resonant frequency on a specific bone or muscle tissues, if applied for certain duration will enhance mobility and performance.
